(19) United States
(12) Patent Application Publication
(10) Pub. No.: US 2022/0273682 A1
(43) Pub. Date: Sep. 1, 2022

(54) ACEFAPC FOR THE TREATMENT OF ACETYLCHOLINE-DEPENDENT DISEASES

(71) Applicants: LIPTHER, Villeurbanne (FR); INSTITUT NATIONAL DES SCIENCES APPLIQUEES DE LYON, Villeurbanne (FR); UNIVERSITE CLAUDE BERNARD LYON 1, Villeurbanne (FR); INSTITUT NATIONAL DE LA SANTE ET DE LA RECHERCHE MEDICALE, Paris (FR); INSTITUT NATIONAL DE LA RECHERCHE AGRONOMIQUE, Paris (FR)

(72) Inventors: Michel LAGARDE, Decines-Charpieu (FR); Evelyne VERICEL, Villeurbanne (FR); Madeleine PICQ, Chateauneuf (FR); Michel GUICHARDANT, St Didier au Mont d'Or (FR); Nathalie BERNOUD-HUBAC, Morance (FR); Baptiste FOURMAUX, Lyon (FR)

(21) Appl. No.: 17/670,760

(22) Filed: Feb. 14, 2022

Related U.S. Application Data
(63) Continuation of application No. 16/491,492, filed on Sep. 5, 2019, now abandoned.

(30) Foreign Application Priority Data
Mar. 8, 2017 (FR) 1751880

(51) Int. Cl. A61K 31/685; A61K 9/00

(52) U.S. Cl. CPC A61K 31/685; A61K 9/0019; A61K 9/0073

(57) ABSTRACT

The present invention relates to AceFaPC (1-Acetyl-2-Fatty acyl-glyceroPhosphoCholine) for use in the prevention and treatment of diseases associated with an acetylcholine deficiency. The invention also relates to the AceFaPC molecule in which Fa represents an unsaturated acyl comprising at least 14 carbon atoms, and the pharmaceutical compositions comprising same.

### FIELD OF THE INVENTION

The present invention relates to AceFaPC (1-acetyl-2-fatty acyl-glycerophosphocholine) for use in the prevention and treatment of diseases associated with acetylcholine deficiency. The invention also relates to the AceFaPC molecule, for which Fa represents an unsaturated acyl containing at least 14 carbon atoms, and pharmaceutical compositions containing same.

### STATE OF THE ART

AceDoPC (1-acetyl,2-docosahexaenoyl-phosphatidylcholine) is a docosahexaenoic acid (DHA) transporter, well known to those skilled in the art, whose enzymatic synthesis is described in application WO 2008/068413. It is particularly known as a modulator of platelet activation by PAF (WO2013037862A1). It has also been shown that the passage of a reconstituted blood-brain barrier is promoted with AceDoPC, compared with non-esterified DHA or PC-DHA (Hashem M. et al., Mol. Neurobiol. 2016; Bernoud-Hubac N. et al., OCL, 2017). Another study showed that AceDoPC used as DHA transporter in the brain prevented the spread of brain lesions when injected into rats after ischemic stroke (Chauveau et al. Curr Neurovasc Res. 2011; 8: 95-102 and Lagarde M. et al., OCL 2016, 23(1) D102). While only the treatment of ischemia has been studied in vivo in a rat model, the use of AceDoPC as a supplier of DHA to the brain is considered in relation to neurological diseases associated with DHA deficiency (Hachem M. et al., Mol Neurobiol., 2016, 53(5), 3205-15). The link between DHA and the prevention of Alzheimer's disease has been mentioned, particularly the fact that Alzheimer's patients have a DHA deficiency. Thus, a DHA transporter such as AceDoPC could be considered to help prevent Alzheimer's disease but not to treat it. This assumption, made in relation to DHA transport, cannot be extended generally to an AceFaPc molecule when the fatty acid is not DHA.

While the studies focused on DHA transport and supply, the inventors have now demonstrated that AceDoPC and, generally, the AceFaPCs, for which Fa is an acyl radical of unsaturated fatty acid of at least 14 carbon atoms, can quickly transfer its acetyl group to a substrate comprising an alcohol, in particular primary.

Based on this observation, the inventors considered AceDoPC no longer as a carrier of DHA, but as a supplier of acetyl. Then, since AceDoPC also includes a choline group, the inventors considered the possibility of producing acetylcholine from AceDoPC, in environments that are poor in sources of choline and/or acetyl. AceDoPC can also acetylate the thiol of coenzyme A (HSCoA) to give acetyl-CoA, precursor of acetylcholine in the physiological reaction Acetyl-CoA+choline→acetylcholine+HSCoA.

This has been confirmed by various experiments that suggest that AceDoPC and, more generally, the AceFaPCs can be used to treat diseases associated with acetylcholine deficiency, independently of any DHA supply.

### DISCLOSURE OF THE INVENTION

The present invention therefore relates to AceFaPC of general formula (I)

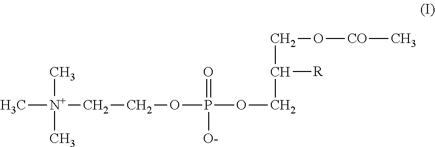

wherein R represents the acyl radical of an unsaturated fatty acid containing at least 14 carbon atoms, for use in the prevention and treatment of diseases associated with acetylcholine deficiency.

The invention also concerns an AceFaPC of formula (I′)

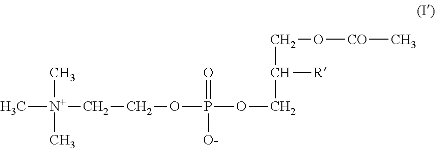

wherein R′ represents the acyl radical of an unsaturated fatty acid containing at least 14 carbon atoms with the exception of the acyl radical of DHA, hydrates, pharmaceutically acceptable salts or pharmaceutically acceptable solvates.

The invention also relates to a mixture of AceFaPC of formula (I′) and AceDoPC, as well as a pharmaceutical composition comprising an AceFaPC of formula (I′) alone or in mixture with AceDoPC and an appropriate excipient for its administration.

### DETAILED DESCRIPTION OF THE INVENTION

The present invention therefore relates to AceFaPC of general formula (I)

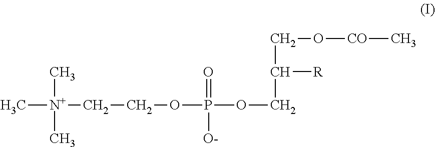

wherein R represents the acyl radical of an unsaturated fatty acid containing at least 14 carbon atoms, hydrates, pharmaceutically acceptable salts or pharmaceutically acceptable solvates.

It relates to these AceFaPCs of formula (I) for use in the prevention and treatment, more particularly the treatment, of diseases associated with acetylcholine deficiency.

The invention also relates to a method for the prevention and treatment of a disease associated with acetylcholine deficiency in a patient which comprises administering to said patient an appropriate dose of AceFaPC of formula (I) or a mixture of AceFaPC of formula (I).

The invention is particularly suitable for the prevention and treatment, and more particularly the treatment, of these diseases in humans. In particular, treatment will be used when the patient has been identified as having an acetylcholine deficiency or is likely to develop such a deficiency.

Diseases associated with acetylcholine deficiency include
Alzheimer's disease associated with acetylcholine deficiency in the brain,
diseases of neuromuscular transmission in which acetylcholine deficiency is recognized, notably neuromuscular diseases, in particular myopathies with acetylcholine deficiency.

“Acetylcholine deficiency” means that the amount of acetylcholine measured in an organ of an individual is much lower than the normal expected in an individual who does not have this deficiency (or healthy individual). This substantial decrease compared with a healthy individual leads to a metabolic imbalance or dysfunction of the organ.

The invention therefore relates to AceFaPC of formula (I) or of formula (I′), or a mixture of AceFaPC of formula (I), for use in therapy, more particularly for the treatment of diseases associated with acetylcholine deficiency in a patient for whom the presence of such acetylcholine deficiency has been previously identified.

The invention also relates to a method for the treatment of a disease associated with acetylcholine deficiency in a patient which comprises

a) selecting patients in whom acetylcholine deficiency has been identified, and

b) administering to said patient an appropriate dose of AceFaPC of formula (I) or (I′) or a mixture of AceFaPC of formula (I).

According to a preferred embodiment of the invention, AceFaPC must be administered in such a way that it is substantially “intact” when it reaches the target organ in which acetylcholine must be produced to prevent or compensate for its deficiency. Substantially “intact” means that a sufficient amount of AceFaPC reaches said organ without having been modified, in particular by hydrolysis of the acetyl.

It has been observed that acetyl loss was favored in the mammalian gastrointestinal tract (WO 2017/006047). Therefore, the preferred modes of administration will be those that are appropriate to avoid the gastrointestinal tract. These include administration by the intravenous, intramuscular, subcutaneous, transdermal and inhalation routes.

For AceFaPC of formula (I), where R represents the acyl radical of an unsaturated fatty acid containing at least 14 carbon atoms, the unsaturated fatty acid containing at least 14 carbon atoms is advantageously a fatty acid of more than 18 carbon atoms and up to more than 22 carbon atoms, in particular 16 18, 20, 22 and 24 carbon atoms. These unsaturated fatty acids are preferentially polyunsaturated. These unsaturated fatty acids are well known to those skilled in the art.

They are particularly selected from palmitoleic, oleic, linoleic (LA), alpha- or gamma-linolenic (ALA or GLA), arachidonic (ARA), eicosapentaenoic (EPA), dihomo-gamma-linolenic, docosahexaenoic (DPA), erucic and nervonic acids.

Preferably, the radical R of AceFaPC of formula (I) is the acyl radical of a polyunsaturated fatty acid selected from oleic (OL), linoleic (LA), alpha- or gamma-linolenic (ALA or GLA), arachidonic (ARA), eicosapentaenoic (EPA) and docosahexaenoic (DHA) acids. Preferably, the R of AceFaPc of formula (I) is the acyl radical of a polyunsaturated fatty acid selected from arachidonic acid (ARA) and docosahexaenoic acid (DHA). These preferred products are called AceArPC and AceDoPC, respectively.

For AceFaPC of formula I′, R′ is advantageously chosen from the acyl of the fatty acids defined above with the exception of DHA.

“Hydrates” refers to a compound in hydrated form. Examples include semi-hydrates, monohydrates and polyhydrates.

The salts of compounds of formula (I) or of formula (I′) according to the present invention include those with acids or bases, depending on the substituents present. These include pharmaceutically acceptable salts, such as sodium, potassium and calcium salts.

“Solvents” means a form of the compound associated with one or more solvent molecules, in particular used during its synthesis or purification, but not in solution in the latter. The solvent in question will be pharmacologically acceptable.

According to the invention, “appropriate dose” or “appropriate amount” means any amount that increases the amount of acetylcholine and preferably restores an amount of acetylcholine close to the normal expected in a healthy individual. This appropriate dose can be taken once or several times, with repeated doses over time. Insofar as the treated disease is a chronic condition, treatment can be taken for the patient's entire life, with appropriate doses adjusted according to the progression of the disease.

For the prevention and treatment of diseases associated with acetylcholine deficiency, those skilled in the art may choose to use an AceFaPC alone or a mixture of AceFaPC. They may also combine AceFaPC or the mixture of AceFaPC with standard treatments for diseases associated with acetylcholine deficiency.

The invention also relates to a mixture of AceFaPC of formula (I) defined above comprising at least two AceFaPCs for which the radicals R are different, in all proportions. According to a preferred embodiment of the invention, at least one of the AceFaPCs in the mixture is AceDoPC, the molecule of formula I for which R is the acyl radical of DHA. A preferred mixture according to the invention is therefore a mixture comprising AceDoPC and at least one AceFaPC of formula (I′) wherein R is the acyl radical of a polyunsaturated fatty acid selected from oleic (OL), linoleic (LA), alpha- or gamma-linolenic (ALA or GLA), arachidonic (ARA) and eicosapentaenoic (EPA) acids, preferably ARA, in all proportions.

The preparation of AceFaPCs is known to those skilled in the art, in particular according to the method described in applications WO 2008/068413 or WO 2017/006047. Mixtures of AceFaPC according to the invention can be prepared by mixing two purified AceFaPCs, or by preparing the AceFaPCs from a source of unsaturated phosphatidylcholines comprising a mixture of unsaturated fatty acids, for example a mixture of DHA and ARA in predetermined proportions.

The AceFaPC mixture according to the invention may include more than two different AceFaPCs, especially when the source of unsaturated phosphatidylcholines includes a mixture of more than two unsaturated fatty acids.

The sources of unsaturated fatty acids useful for the preparation of AceFaPCs according to the invention, and in particular AceFaPC mixtures, are well known to those skilled in the art. An example is egg yolk phosphatidylcholines comprising at the sn-2 position 60% oleoyl (18:1), 30% linoleoyl (18:2), 8% arachidonoyl (20:4) and 2% docosahexaenoyl (22:6) (https://kewpie.co.jp).

Advantageously, the weight ratio of the first AceFaPC to the second AcFaPC ranges from 1/99 to 99/1. Especially when the first AceFaPC is AceDoPC, the AceDoPC content in the mixture may range from 1 to 10 wt % or more, the AceDoPC content in the mixture which may depend both on the source of unsaturated phosphatidylcholines and its DHA content, and on any steps of purifications and of concentration of the mixture.

The following relative proportions of fatty acid acyls will be advantageously present in an AceFaPC mixture according to the invention

| Acyl | Relative % |
|---|---|
| Oleyl | 10-80 |
| Linoleoyl | 5-50 |
| Linolenoyl | 0-5 |
| Arachidonoyl | 0-10 |
| Eicosapentaenoyl | 0-10 |
| Docosahexaenoyl | 0-10 |

According to a particular embodiment of the invention, the relative docosahexaenoyl content in this mixture may range from 1% to 10%.

The invention also relates to a combination product, or “kit of parts”, for simultaneous or time-delayed use, which comprises on the one hand AceDoPC and on the other hand at least one AceFaPC of formula (I) which is not AceDoPC, as defined above.

The invention also relates to a pharmaceutical composition comprising at least one mixture of AceDoPC and at least one AceFaPC of formula (I′) (AceFaPC of formula (I) which is not AceDoPC) and at least one pharmaceutically acceptable excipient.

Those skilled in the art are familiar with the pharmaceutically acceptable excipients that may be used for the preparation of a pharmaceutical composition, in particular those described in the pharmacopeial standards. They will preferably choose excipients that will preserve the structure of the AceFaPCs for their storage, in particular to prevent hydrolysis of the sn-1 position resulting in loss of the acetyl.

The pharmaceutical compositions are preferably in a form suitable for administration by the intravenous, intramuscular, subcutaneous, transdermal or inhalation route.

### DESCRIPTION OF THE FIGURES

FIG. 1 describes the synthesis of acetylcholine from AceFaPC under the action of a phospholipase D.

FIG. 2 shows the detection of acetylcholine and its main fragmentation product by mass spectrometry.

FIG. 3 shows the percentages of passage from blood to the brain (rat) based on radioactive DHA, as a function of time. (Hashem et al. Mol. Neurobiol. 2016)

### EXAMPLES

Several experiments were conducted using AceFaPC (DHA and oleic acid) as a possible precursor of acetylcholine and final measurement of the latter by radiochromatography or mass spectrometry.

### Example 1: Synthesis of Acetylcholine from AceFaPC with Rat Brain Homogenate

Rat brain homogenate was incubated with AceFaPC, labeled with ¹⁴C on the acetyl, in Tris-HCl pH 8 buffer in the presence of an anti-protease cocktail and an acetylcholine esterase inhibitor. Incubation takes place at 37° C. for one hour. After extraction with an ethanol/chloroform mixture, the organic phase and the aqueous phase are separated and analyzed. After separation by thin-layer chromatography, the products are visualized with a radioactivity reader. A radioactive spot corresponding to the migration of acetylcholine is detected. Incubation of rat brain homogenate with labeled AceFaPC therefore allows the synthesis of radioactive acetylcholine, after release of choline by cerebral phospholipase D and chemical coupling of this choline with a labeled acetyl group in the sn-1 position of AceFaPC and/or coupling of the radioactive acetyl provided by AceFaPC and endogenous choline.

### Example 2: Synthesis of Acetylcholine from AceFaPC with a Phospholipase D

Acellular incubation of AceFaPC was performed in the presence of a microbial (Streptomyces chromofuscus) phospholipase D in a Tris-HCl pH 8 (brain pH) buffer for one hour at 37° C. Following incubation, phospholipase D was destroyed by adding ethanol. After centrifugation, the aqueous ethanolic mixture was separated and evaporated to dryness. The residue was dissolved in the mixture 95% acetonitrile/5% ammonium formate and then filtered by centrifugation. Acetylcholine was detected in the solution by mass spectrometry. Acetylcholine was detected on the basis of both its molecular mass (146) and that of its main fragmentation product (87: majority ion corresponding to the radical CH₃—COO—CH₂—CH₂—) (FIG. 2).

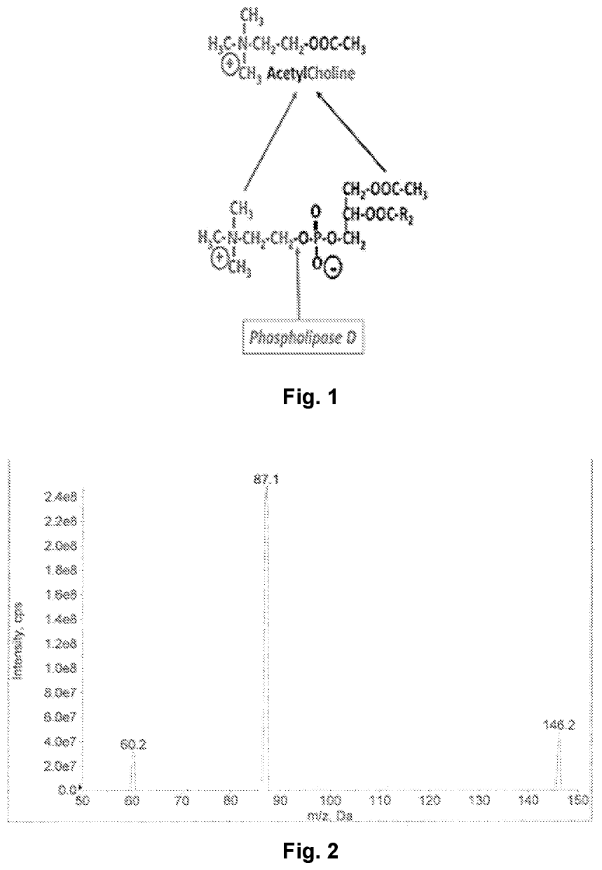

In these acellular experiments, two substrates were used considering the lowest unsaturation for acyl in the sn-2 position, namely the oleoyl radical (R₂—COO—, FIG. 1): AceOIPC and the highest with the docosahexaenoyl radical: AceDoPC®. For AceOIPC, approximately 5% of the released choline was converted to acetylcholine. For AceDoPC®, this conversion reached 36%. The reasons for this difference are unknown at this stage of the experiment. However, it can be hypothesized that the conjugation of choline with the acetyl radical is better if the sn-2 position is less hindered, which is the case for the docosahexaenoyl radical (with 6 double bonds) compared with the oleoyl radical (with a single double bond) because the folding of the acyl chain with 6 double bonds is much greater than for a single double bond. This result is particularly encouraging for AceFaPC containing polyunsaturated acyls, such as docosahexaenoyl and arachidonoyl (not yet tested), the two most important acyls in the brain.

Acellular incubation of AceFaPC was also performed in the presence of an equimolecular amount of choline chloride dissolved in Tris-HCl pH 8 buffer for one hour at 37° C. with shaking. After evaporation to dryness under nitrogen, the residue was dissolved in the mixture 95% acetonitrile/5% ammonium formate. As previously described, after centrifugal filtration, acetylcholine was measured by mass spectrometry.

These experiments show that incubations of AceFaPC in the presence of phospholipase D (which releases choline from AceFaPC) or in the presence of exogenous choline allow the synthesis of acetylcholine.

The proximity of the two constituent groups (acetyl and choline) of acetylcholine on the same molecule, approximately one nm apart, is such as to facilitate their conjugation into an end product with respect to the distance within the same cell (distance in the pm range) as is accepted in all biochemical processes concerned by this proximity of reactants.

This proximity is summarized by the reaction shown in FIG. 1.

The results confirm the hypothesis of the mechanism of action of transformation of AceFaPC into acetylcholine in the brain under the action of cerebral phospholipase D.

### Example 3: AceDoPC Transport in Rat Brain (Hashem et al. 2016)

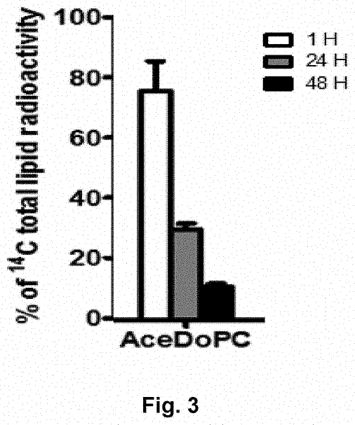

AceDoPC labeled with ¹⁴C on the docosahexaenoyl residue was injected into the bloodstream of various rats. After 1 h, 24 h and 48 h, the rat brains were analyzed to locate the radioactivity in different lipid compartments. For AceDoPC, its radioactivity decreased from 80% of the total injected after 1 hour, then 30% after 24 hours and 10% after 48 hours (FIG. 3). These results confirm that AceDoPC injected into the bloodstream is substantially “intact” when it reaches the brain where it is metabolized, notably by the loss of its acetyl residue with the formation of acetylcholine.

### REFERENCES

WO2013037862

WO 2008/068413

Bernoud-Hubac N. et al., OCL 2017, 24(2) D205

Hashem M. et al., Mol Neurobiol. 2016, 53(5), 3205-15

Lagarde M. et al., OCL 2016, 23(1) D102

1.-12. (canceled)

13. A method for the prevention and the treatment of a disease associated with acetylcholine deficiency in a patient in need thereof, said method comprising administering to said patient an appropriate dose of AceFaPC of general formula (I) or a mixture of AceFaPC of general formula (I) 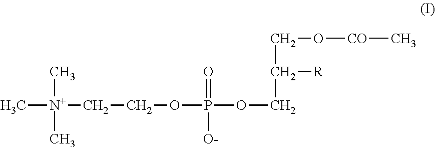
wherein R represents the acyl radical of an unsaturated fatty acid containing at least 14 carbon atoms, hydrates, pharmaceutically acceptable salts or pharmaceutically acceptable solvates.

14. The method according to claim 13, wherein said disease associated with acetylcholine deficiency is selected from Alzheimer' s disease and neuromuscular diseases.

15. The method according to claim 13, wherein the AceFaPC of general formula (I) or the mixture of AceFaPC of general formula (I) is administered by a suitable route to avoid the gastrointestinal tract.

16. The method according to claim 15, wherein the AceFaPC of general formula (I) of the mixture of AceFaPC of general formula (I) is administered by the intravenous, intramuscular, subcutaneous, transdermal or inhalation route.

17. The method according to claim 13, wherein the mixture of AceFaPC of formula (I) comprises a first molecule of AceFaPC of formula (I) wherein R is a first acyl radical of an unsaturated fatty acid containing at least 14 carbon atoms and at least a second molecule of AceFaPC of formula (I) wherein R represents an acyl radical of an unsaturated fatty acid containing at least 14 atoms and which is different from the acyl radical of the first AceFaPC.

18. The method according to claim 13, wherein the AceFaPC mixture comprises a first molecule of AceFaPC of formula (I) wherein R is the acyl radical of DHA (AceDoPC) and at least one molecule of AceFaPC of formula (I) wherein R represents an acyl radical of an unsaturated fatty acid containing at least 14 carbon atoms and which is not the acyl radical of DHA.

19. The method according to claim 18, wherein the unsaturated fatty acid of the second molecule of AceFaPC of formula (I) is selected from oleic (OA), linoleic (LA), alpha- or gamma-linolenic (ALA or GLA), eicosapentaenoic (EPA), and arachidonic (ARA) acids.

20. The method according to claim 19, wherein the unsaturated fatty acid of the second AceFaPC is arachidonic acid (ARA).

21. The method according to claim 17, characterized in that the relative proportions of fatty acid acyls in the different AceFaPCs of formula (I) are as follows:

| Acyl | Relative % |
|---|---|
| Oleyl | 10-80 |
| Linoleoyl | 5-50 |
| Linolenoyl | 0-5 |
| Arachidonoyl | 0-10 |
| Eicosapentaenoyl | 0-10 |
| Docosahexaenoyl | 0-10 |

22. The method according to claim 13, wherein the AceFaPC of general formula (I) or the mixture of AceFaPC of general formula (I) is in a form of a pharmaceutical composition comprising at least one pharmaceutically acceptable excipient.

23. The method according to claim 17, wherein the molecules of the mixture are administered simultaneously or separately over time.

24. The method according to claim 21, wherein the docosahexaenoyl content ranges from 1% to 10%.
